(19) United States
(12) Patent Application Publication
(10) Pub. No.: US 2009/0083067 A1
(43) Pub. Date: Mar. 26, 2009

(54) Methods and kits for selecting a health care product

(76) Inventors: Mary Elaine Freeland, Loveland, OH (US); Nancy Jean McCarthy, Cincinnati, OH (US); Susan Louise Abeln, Cincinnati, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/157,399

(22) Filed: Jun. 10, 2008

Related U.S. Application Data
(63) Continuation-in-part of application No. 11/391,839, filed on Mar. 29, 2006.

(51) Int. Cl. G06Q 50/00

(52) U.S. Cl. 705/2; 604/500

(57) ABSTRACT

A method is provided for selecting and administering a health care product comprising the steps of:
a. providing selection indicia for selection of a health care product;
b. providing corresponding product indicia associated with a containment device containing a health care product, wherein a potential user matches selection indicia and product indicia to select an appropriate health care product; and
c. administering the health care product selected.

Kits containing selection indicia, product indicia and one or more health care products, as well as methods of using the kit are also disclosed.

### CROSS REFERENCE TO RELATED APPLICATIONS

This application is a Continuation-In-Part of U.S. non-provisional application Ser. No. 11/391,839 filed Mar. 29, 2006.

### FIELD OF THE INVENTION

The present invention is directed to methods of selecting and administering a health care product. The method relates to providing selection indicia for selection of a health care product and providing corresponding product indicia such that a user can select an appropriate health care product; and administering the health care product selected. Kits containing selection indicia, product indicia and a health care product, as well as methods of using the kits are also disclosed. The method is particularly useful for guiding a user to an appropriate health care product.

### BACKGROUND OF THE INVENTION

Health care products are widely used and may include products chosen by a user without the assistance of a health care professional or chosen as the result of a recommendation or prescription from a health care professional. Regardless of the route by which a particular product is chosen or used, as health professionals are not always readily available, self-assessment of symptoms and treatment is an important aspect of obtaining an appropriate health care product and providing ongoing compliance with use of the product. Users may not wish to continue use of products that are not immediately effective to treat the particular health condition at issue. There may be various reasons a product is not immediately effective. The product may simply take a period of time to become effective, or a user may not have the appropriate product, or strength of product.

As but one example, health care product users experiencing functional digestive conditions (including irritable bowel syndrome or temperamental bowel syndromes, constipation dominant, diarrhea dominant, or alternating constipation/diarrhea) may have a wide range of symptoms. These symptoms may occur often, but the precise frequency and severity may be unpredictable, resulting in stress, a feeling of lack of control, all of which may significantly impact the user's quality of life. In addition, these users are often disappointed with the medical care received, as certain functional disorders are by nature diagnoses of exclusion and these users are often told that nothing is wrong with them.

Due to the episodic nature of certain health conditions, it may be difficult for a user of a health care product to assess what product may be appropriate and later, whether symptom alleviation is due to use of the product or whether the user is simply in a remission phase of his or her condition. Moreover, efficacy of certain health care products may commence only after a period of time, for example weeks or months, and the user needs a tool that enables selection of an appropriate product initially and encourages continued use of the product even if positive results are not immediately seen.

Thus there remains a need for methods of selecting and administering a health care product that is useful for a user of a health care product to self-assess the appropriate product to use, and to self-assess the efficacy of the product in relation to a particular health condition.

### SUMMARY OF THE INVENTION

The present invention is directed to methods of selecting and administering a health care product. In particular, the method comprises the steps of:
a. providing selection indicia for selection of a health care product;
b. providing corresponding product indicia associated with a containment device containing a health care product, wherein a potential user matches selection indicia and product indicia to select an appropriate health care product; and
c. administering the health care product selected.

Kits comprising selection indicia, product indicia and a health care product, as well as methods of using the kits are also disclosed. These and other embodiments of the invention are described herein.

### BRIEF DESCRIPTION OF THE FIGURES

FIG. 1 demonstrates a non-limiting example of a compliance aid of the present invention. The figure is more particularly described at Example 1.

FIG. 2 demonstrates a non-limiting example of a compliance aid of the present invention. The figure is more particularly described at Example 2.

FIGS. 3a and 3b demonstrate a non-limiting example of a compliance aid of the present invention. The figures are more particularly described in Example 3.

FIGS. 4a and 4b demonstrate a non-limiting example of a compliance aid of the present invention. The figure is more particularly described in Example 4.

FIG. 5 demonstrates a non-limiting example of a portion of a compliance aid of the present invention. The figure is more particularly described in Example 5.

FIG. 6 demonstrates a non-limiting example showing selection indicia and product indicia of the present invention. The figure is more particularly described in Example 6.

FIG. 7 demonstrates a non-limiting example showing selection indicia and product indicia of the present invention. The figure is more particularly described in Example 7.

### DETAILED DESCRIPTION OF THE INVENTION

Various documents including, for example, publications and patents, are recited throughout this disclosure. All such documents are hereby incorporated by reference.

Trade names for products or components including various ingredients may be referenced herein. The inventors herein do not intend to be limited by materials under a certain trade name.

In the description of the invention various embodiments or individual features are disclosed. As will be apparent to the ordinarily skilled practitioner, all combinations of such embodiments and features are possible and can result in preferred executions of the present invention.

The compositions herein may comprise, consist essentially of, or consist of any of the elements as described herein.

While various embodiments and individual features of the present invention have been illustrated and described, various other changes and modifications can be made without departing from the spirit and scope of the invention. As will also be apparent, all combinations of the embodiments and features taught in the foregoing disclosure are possible and can result in preferred executions of the invention.

### Compliance Aids of the Present Invention

The present invention relates in part to a compliance aid suitable for use in conjunction with a health care product, as well as kits comprising the compliance aid and methods of using the compliance aid. The aid is particularly useful for motivating a user to continue use of the product, communicating results of the product to the user, or the like. The compliance aids described herein comprise:
(a) a reference to a health care product that is useful for the treatment of a health condition;
(b) a plurality of symbolic data entry fields; and
(c) information related to use of the compliance aid, wherein the information comprises direction to a user to:
   i. on a periodic basis, enter symbolic data into at least one of the symbolic data entry fields, wherein entry of the symbolic data is based on perceived symptoms of the health condition at or near the time of data entry; and
   ii. self-assess treatment of the health condition based on visual review of a plurality of entries of the symbolic data in the symbolic data entry fields.

A user can also self-determine one or more appropriate health care products to use singly or in combination, or continue using, based on entries in the compliance aid. The compliance aid can also comprise indicia corresponding to selection and/or product indicia to aid a user in selecting one or more appropriate health care products.

The compliance aid may be provided in a variety of forms including, for example, paper, computers, personal digital assistants, telephones (including cellular phones and other communication devices), BLACKBERRY or other devices uses for communicating voice or text), internet, and the like.

Non-limiting descriptions of the various components of the compliance aid are as follows:

The users herein include, but are not limited to, humans. The human user is often the user of the compliance aid and of the health care product referenced by the compliance aid. However, it will be recognized that a human may use the compliance aid, but for the purpose of tracking progression or alleviation of symptoms experienced by a non-human animal, such as a domestic animal (e.g., a dog, cat, rabbit, horse, or the like). In such instances, the user of the compliance aid is still the human, while the non-human animal experiences the health condition.

### Reference to the Health Care Product

The compliance aid comprises a reference to a health care product that is useful for the treatment of a health condition. As such, it is not necessary that the compliance aid is directly provided to a user with the health care product (although, in one embodiment as described below, the kits of the present invention comprise the compliance aid and a health care product). Rather, the compliance aid need only refer to the health care product that is being used in conjunction with the compliance aid. The reference may be general (e.g., “use this aid concurrently or around the time of use of a product suitable for the treatment of digestive balance disorders or the treatment of irritable bowel syndrome”) or specific (e.g., “use this aid concurrently or around the time of use of ALIGN™, a product containing a bacterial microorganism”). The reference to the health care product therefore provides context for use of the compliance aid, so that the user can associate use of the health care product with progression or alleviation of symptoms.

The health care product is unlimited in terms of the health condition that it is used to treat, and unlimited in the form in which the health care product can be administered. Non-limiting examples of common product and dosage forms suitable for use with health care products of the present invention include dosage forms selected from the group consisting of: capsule, chewable tablet, swallowable tablet, buccal tablet, troche, powder, lozenge, soft chew, solution, suspension, spray, tincture, decoction, infusion, syrup, exlixir, wafer, food product, and combinations thereof.

In one embodiment, particularly as the compliance aid may be useful for understanding symptom progression or alleviation in a self-treated user, the health care product may be a product that is used with relatively little or no input from a health care professional (such as a pharmacist, nurse, physician, veterinarian, or managed care organization). In such cases, the user may be consulting with a health care professional on an infrequent basis. For purposes of this invention, these health care products may be referred to as over-the-counter health care products, which are generally those products available to a user without the need of a prescription from a health care professional, and may range from traditional medicines to dietary supplements to functional foods that may be marketed with a health claim. In some geographies, such over-the-counter health care products are in practice obtained directly by the user at, for example, retail stores, pharmacies, wholesale cooperatives, internet channels, mail order, hospitals, nursing homes, and the like. In other geographies, such over-the-counter health care products are in practice obtained through a pharmacist or like professional through simple request.

In yet other embodiments, the health care product may be a product that is used with moderate or significant input from a health care professional. In such cases, the user may use the compliance aid to report progression or alleviation of symptoms to the professional such that ongoing treatment may be rendered more successful. For purposes of this invention, these health care products may be referred to as prescription health care products, which are generally those products available to a user through a prescription from a health care professional. In most geographies, such prescription health care products are in practice obtained directly by the user at, for example, retail stores, pharmacies, wholesale cooperatives, internet channels, mail order, hospitals, nursing homes, and the like, but only upon demonstration of a prescription from the relevant health care professional.

In one embodiment herein, the health care product is useful for treating a condition wherein it may be difficult to associate symptom relief to product use. For example, the health care product may be selected from respiratory health care products, insomnia products, and arthritis products.

In one embodiment herein, the health care product is useful for treating a life-stage change (for example, onset of menopause or menstruation), wherein it may be difficult to assess which interventions are having an impact on the quality of life associated with the change.

In one embodiment herein, the health care product is useful for chronic conditions in which relief is either not immediate or cannot be objectively measured. Such conditions may include depression, chronic fatigue syndrome, gastrointestinal health conditions, respiratory conditions, pain (including, for example, chronic lower back pain, migraine), fibromyalgia, arthritis, urinary incontinence, sexual dysfunction, and the like.

In another embodiment herein, the health care product is useful for episodic conditions in which recur periodically. Such conditions include, for example, premenstrual syndrome, or allergies.

In one embodiment herein, the health care product is useful for improving and maintaining overall health and well-being, which also can not necessarily be immediately or objectively measured. Non-limiting examples of aspects of overall health and well-being include improving and maintaining joint health, immune health, respiratory health, digestive and gastrointestinal health, skin health, hair health, eye health, bone health, heart and cardiovascular health, respiratory health, sleep health, mood, stress reduction, energy level, and combinations thereof. Other aspects of health for which a health care product of the present invention can be useful include satiety, weight loss, and body weight management.

Non-limiting examples of health care products suitable for improving and maintaining overall health and well-being include vitamins, minerals, metals, anti-oxidants, carotenoids, fiber, prebiotics, resistant starch, plants and plant-derived materials, and combinations thereof. Such products can be administered in the dosage forms and amounts commonly recommended for such products.

In another embodiment herein, the health care product is useful for detoxifying and/or cleansing the digestive system and/or colon; reducing the risk of colon cancer; protecting the throat, teeth and gums from stomach acids; and repairing and/or preventing further damage to the digestive system.

In one embodiment herein, the health care product is selected from gastrointestinal health care products and respiratory health care products.

The compliance aids herein are particularly useful in reference to a gastrointestinal health care product. Gastrointestinal health care products are useful for the treatment of gastrointestinal tract conditions, which may present symptoms that are episodic in nature. Wherein symptoms are episodic, the user of the product may have difficulty discerning symptom improvement due to use of the product itself, or whether the user is in a remission phase of the condition. In such instances, the compliance aid is particularly useful as the user documents general trends associated with symptom progression or alleviation, or user experiences by which episodes, including flares, are triggered (such as, for example, ingestion of a specific type of food, ingestion of food outside residence (e.g., “eating out”), travel, use of another health care product (e.g., contraindicated products), stressful condition, failure to administer the health care product, menstruation, and combinations thereof).

For convenience, gastrointestinal health care products may be categorized as those useful for the treatment of lower gastrointestinal tract conditions or upper gastrointestinal tract conditions. However, some products may be useful for the treatment of both, and descriptions and categorizations herein are of course provided merely for convenience and are intended as non-limiting.

Lower gastrointestinal tract conditions include, but are not limited to, functional digestive disorders (including but not limited to irritable bowel syndrome, temperamental digestive systems, constipation dominant, diarrhea dominant, and alternating constipation/diarrhea), inflammatory bowel disease, constipation, diarrhea (including traveler's diarrhea), bloating, flatulence, abdominal cramping, abdominal pain, gas, Crohn's Disease, ulcerative colitis, diverticulitis, microscopic colitis, diverticular disease, dyspepsia, small intestinal bacterial overgrowth, lactose intolerance, celiac disease, and the like. In a particular embodiment herein, gastrointestinal health care products suitable for the treatment of a lower gastrointestinal tract conditions is particularly interesting herein.

Non-limiting examples of gastrointestinal health care products suitable for the treatment of lower gastrointestinal tract conditions include PEPTO-BISMOL® (containing bismuth subsalicylate and commercially available from The Procter & Gamble Company) and other bismuth-containing products, ALIGN® (containing a bacterial microorganism (e.g., BIFANTIS® (a strain of Bifidobacterium infantis, commercially available from The Procter & Gamble Company), ACTIVIA® (containing a bacterial microorganism, commercially available from The Groupe Danone), ASACOL® (containing mesalamine and commercially available from The Procter & Gamble Company).

In one embodiment of the invention, the health care product (whether gastrointestinal health care product or otherwise) comprises a bacterial microorganism, for example a probiotic. Preferably, the bacterial microorganism is a lactic acid bacteria. Non-limiting examples of lactic acid bacteria include strains of Streptococcus lactis, Streptococcus cremoris, Streptococcus diacetylactis, Streptococcus thermophilus, Lactobacillus bulgaricus, Lactobacillus acidophilus, Lactobacillus helveticus, Lactobacillus bifidus, Lactobacillus casei, Lactobacillus lactis, Lactobacillus plantarum, Lactobacillus rhamnosus, Lactobacillus delbruekii, Lactobacillus thermophilus, Lactobacillus fermentii, Lactobacillus salivarius, Bifidobacterium longum, Bifidobacterium infantis, Bifidobacterium bifidum, and Pediococcus cerevisiae, or mixtures thereof, in particular Lactobacillus salivarius, Bifidobacterium infantis, or mixtures thereof.

As a non-limiting example, strains of Bifidobacterium isolated from resected and washed human gastrointestinal tract as disclosed in WO 00/42168 may be used. An example includes Bifidobacterium infantis strain designated UCC35624, described as being deposited at the National Collections of Industrial and Marine Bacteria Ltd (NCIMB) on Jan. 13, 1999, and accorded the accession number NCIMB 41003.

Embodiments of the methods and kits can provide one or more probiotics at various amounts, to provide various benefits. The probiotic can be administered in various amounts over various periods of time in various dosing regimens such as those described in U.S. patent application Ser. No. 12/056,702.

Non-limiting examples of such amounts include loading amounts of a probiotic of from about 1×10³ to about 1×10¹⁴ cfu of probiotic per day; alternatively from about 1×10⁵ to about 1×10¹⁴ cfu of probiotic per day; and alternatively from about 1×10⁹ to about 1×10¹² cfu of probiotic per day.

Non-limiting examples of such amounts also include maintenance amounts of a probiotic of from about 1×10³ to about 1×10¹² cfu of maintenance probiotic per day; and alternatively from about 1×10⁷ to about 1×10¹⁰ cfu of maintenance probiotic per day. The probiotic used to provide loading amounts of probiotic and that used to provide maintenance amounts of probiotic can be the same or different probiotics, or can be combinations of probiotics.

Upper gastrointestinal tract conditions include, but are not limited to, gastroesophageal reflux disease (GERD), erosive esophagitis, gastroparesis, gastritis, gastric ulcers, duodenal ulcers, heartburn (including frequent heartburn), functional dyspepsia, indigestion, upset stomach, posterior laryngitis, hypersecretory conditions, such as Zollinger-Ellison syndrome, multiple endocrine adenomas and systemic mastocytosis, and the like.

Non-limiting examples of gastrointestinal health care products suitable for the treatment of a upper gastrointestinal tract conditions include PRILOSEC OTC® (containing omeprazole magnesium and commercially available from The Procter & Gamble Company) and other products containing compounds of the class generally known as proton pump inhibitors.

The compliance aids herein are particularly useful in reference to a respiratory health care product. Respiratory health care products are useful for the treatment of respiratory tract conditions which, like gastrointestinal tract conditions, may also present symptoms that are episodic in nature.

Respiratory tract conditions include, but are not limited to, common cold, cough, influenza, allergy, asthma, pulmonary bacterial or fungal infections, COPD, Cystic Fibrosis, pneumonia (viral or bacterial), SARS, respiratory cancers, chronic immunosuppression, and disorders pursuant to respiratory surgeries (pre- and post-operative management). Common cold, cough, influenza, allergy, and asthma (and particularly common cold, cough, and influenza) are particularly interesting herein.

Non-limiting examples of respiratory health care products suitable for the treatment of respiratory tract conditions include any product commercially available under the VICKS®, NYQUIL®, or DAYQUIL® trade names (all commercially available from The Procter & Gamble Company, or any product containing pseudoephedrine, phenylephrine, chlorpheniramine, dextromethorphan, diphenhydramine, doxylamine, guaifenesin, or the like. See, for example, U.S. Pat. No. 5,025,019.

### Plurality of Symbolic Data Entry Fields

The compliance aid herein comprises a plurality of symbolic data entry fields. These fields are suitable for entry of symbolic data relating to progression or alleviation of a symptom association with the health condition treated by the health care product. As used herein, “symbolic data” is data that allows visual representation of such progression or alleviation without reliance on full text sentences or moderate to long phrases (e.g., phrases having more than about four words). In one embodiment, at least a portion of the symbolic data is selected from alphanumeric data (i.e., data containing both letters and numbers), alphabetic data (i.e., data containing letters), numeric data (i.e., data containing numbers), color data (i.e., data represented in a color or combination of colors), variable symbolic data (e.g., check marks, “X” marks, slash marks, asterisks, punctuation marks (e.g., “?”, “!”, and the like), geometric symbols, pictures, and the like), and any combinations thereof. In yet another embodiment, at least a portion of the symbolic data is selected from numeric data, color data, variable symbolic data, and any combinations thereof. In yet another embodiment, the symbolic data is numeric data. In yet another embodiment, the symbolic data is color data. In yet another embodiment, the symbolic data is variable symbolic data.

The form of the symbolic data entry fields may be dependent upon the form of the compliance aid. For example, wherein the compliance aid is in the form of paper, the symbolic data entry fields may be in the form of outlined squares or rectangles suitable for entry of symbolic data. As another example, wherein the compliance aid is comprises an electronic database, the symbolic data entry fields may be fields in which symbolic data is typed or otherwise entered.

For example, each symbolic data entry field may represent a day of the month (or a period of days), such that trends related to treatment may be visualized as the month progresses. It will be obvious in this embodiment that the periods of time may be modified as desired.

As the data is symbolic, interpretation and therefore self-assessment of treatment is readily visualized.

In one embodiment, the symbolic data entry fields are visually arranged such that the user may readily self-assess progression or alleviation of symptoms through a single visual review of the symbolic data entries. As a non-limiting illustration, for example, at least a portion of the compliance aid may be in the form of a calendar representing a month of the year. Each day of the month may be represented by a box in traditional calendar form. The user may, for example, enter color data (using, for example a colored sticker or using a colored mark with a pen or other marking device) for each day of the month as appropriate for how the user feels on that particular data. As an example, the user may be guided by the non-limiting key illustrated at Table 1:

TABLE 1

| Color Data Key for | |
|---|---|
| Compliance Aid | What the Color Data Symbolizes |
| Blue = Great! I | Blue is a calming and relaxing color. Blue also |
| completely forgot | signifies being the best (first prize is a “blue |
| about my digestive | ribbon”). Blue is the most universal color as it is |
| upsets! | the color of sea and sky, and symbolizes |
| | confidence, tranquility and calmness. |
| Green = Good - | Green is the color of nature and symbolizes |
| few if any upsets | growth, harmony, renewal and freshness. Green |
| | is the light that means “go”. Green symbolizes |
| | stability. Green is still not perfect - it is the color |
| | of envy or inexperience. |
| Yellow = | Yellow has a dual personality. Yellow is a warm |
| Occasionally | color and promotes energy and is often used as a |
| Bothered - mild to | sign of hope. Yellow is also the color of |
| moderate upsets | “caution” and can mean trouble ahead. Yellow is |
| | considered an unstable and spontaneous color. |
| Orange = Often | Orange is a very hot color and gives the sensation |
| Bothered - moderate | of heat while not being as aggressive or intense as |
| to severe upsets | red. Orange is an invigorating, motivating color |
| | that symbolizes strength and endurance. Orange |
| | demands attention. |

A similar flow of colors could be tied to other compliance aids used for other conditions. Upon completion of the month or other given time period and symbolic data entry in all of the symbolic data entry fields, a user may visualize with a single visual review how symptoms are progressing or alleviated. For example, wherein more yellow, green, or blue color data entries are made near the end of the month or over a series of months or other time periods, the user self-assesses that symptoms are being alleviated through use of the health care product. On the other hand, wherein more orange or yellow color data entries are made near the end of the month or over a series of months or other time periods, or no significant changes are made at all during the month or other time period, the user may self-assess that the treatment is not efficacious or only moderately efficacious. Particularly wherein symptoms are cyclical or subjective in nature, this self-assessment assists the user with an understanding of the success of the treatment and the efficacy of the health care product without the need for reading lengthy prose entries or attempts to remember severity of symptoms over long periods of time. The self-assessment can also assist the user in choosing, for example, an appropriate dose, strength, amount or type of health care product, products or combination of products, based on the color data entries.

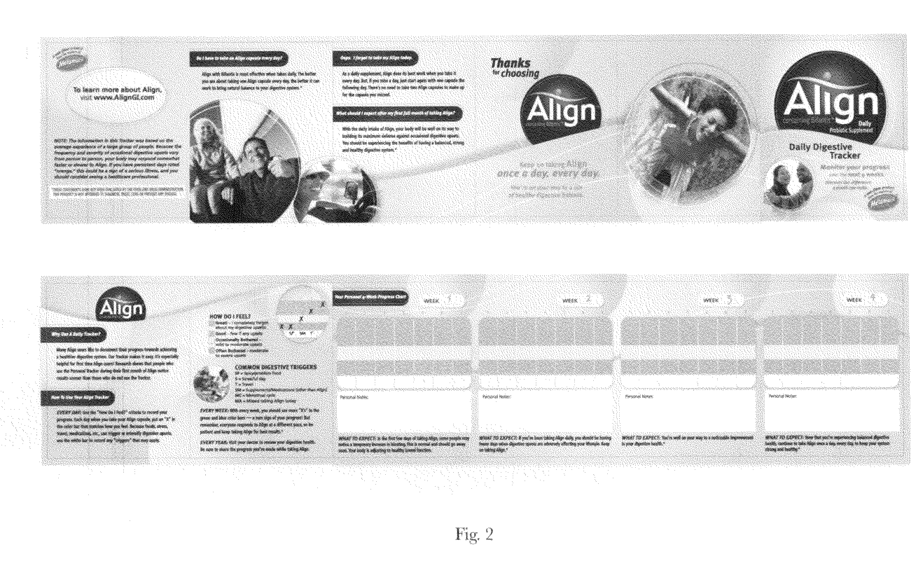

As another example, at least a portion of the symbolic data entry fields comprise a plurality of symbolic data entry sub-fields. To illustrate, a symbolic data entry field representing a specific day of the week (or other period of time) may be divided into such sub-fields, wherein the user enters symbolic data into the sub-field that most closely represents how the user feels about symptoms at the time of data entry. For example, FIG. 2 illustrates symbolic data entry sub-fields. The symbolic data entry fields are color coded as color bars, wherein the color bars are arranged in 28 separate columns corresponding to the 28 symbolic data entry fields. Each color bar within a symbolic data entry field is a symbolic data entry sub-field and are described as follows, arranged in each column from highest to lowest:
Blue=“Great!—I completely forgot about my digestive upsets!”
Green=“Good—few if any upsets”
Yellow=“Occasionally Bothered—mild to moderate upsets”
Orange=“Often Bothered—moderate to severe upsets”
   (Note that the lowest color bar in the column, in this instance the “white” bar, is a symbolic data entry sub-field for entry based on a user experience at or near the time of data entry.)

Such color bars can also include indicia corresponding to selection indicia and/or product indicia, such as:
Blue=“Great!—I completely forgot about my digestive upsets”! Use Align Daily Digestive Care.
Green=“Good—few if any upsets”. Use Align Daily Digestive Care.
Yellow=“Occasionally Bothered—mild to moderate upsets”. Use Align Advanced Daily Digestive Care.
Orange=“Often bothered—moderate to severe upsets”. Use Align Advanced Daily Digestive Care.

### Information Related to Use of the Compliance Aid

The compliance aid further comprises information related to use of the compliance aid, wherein the information comprises direction to a user to:
i. on a periodic basis, enter symbolic data into at least one of the symbolic data entry fields, wherein entry of the symbolic data is based on perceived symptoms of the health condition at or near the time of data entry.

It will be well-understood that the appropriate periodic basis may be dependent upon the particular health condition at issue. For example, wherein the health condition is a gastrointestinal health condition, daily entry of symbolic data may be appropriate such that the user creates a daily record of perception of symptom progression or alleviation. In a non-limiting example, the periodic basis is selected from multiple times daily, daily, multiple times weekly, weekly, multiple times monthly, monthly, and combinations thereof. In yet another non-limiting example, the periodic basis is selected from multiple times daily, daily, weekly, multiple times monthly, and combinations thereof. In yet another non-limiting example, the periodic basis is selected from multiple times daily, daily, weekly, and combinations thereof.

The entry of the symbolic data is based on perceived symptoms of the health condition at or near the time of data entry. Entry based on perceived symptoms at or near the time of data entry is intended to assist with a more accurate recollection of perception of symptom condition. For example, wherein the periodic basis is daily, data entry should correspond with perception of symptom condition on that day. Wherein the periodic basis is less frequent, the user may use personal judgment as to the appropriate time period over which perception of symptoms is assessed for a particular entry. For example, wherein the periodic basis is weekly, the user may reflect on symptom condition during the previous week as the criteria for selecting data entry.

The compliance aid further comprises information related to use of the compliance aid, wherein the information comprises direction to a user to:
ii. self-assess treatment of the health condition based on visual review of a plurality of entries of the symbolic data in the symbolic data entry fields.

As the plurality of data entries are made, the compliance aid enables the user to visually review the entries in order to self-assess progression or alleviation of symptoms (and therefore success of the treatment). For example, depending upon the particular construction of the compliance aid, the self-assessment may be performed through an elevation or descent in particular symbolic data (see e.g., FIG. 2, showing a rising trend of “X” marks) or an abundance, dominance, or general trend of particular symbolic data (e.g., an abundance or dominance of certain colors, numbers, or other types of symbolic data representing a certain symptom condition).

In one embodiment, the compliance aid further comprises direction to a user to, at the discretion of the user, enter symbolic data into at least one of the symbolic data entry fields, wherein entry of the symbolic data is based on a user experience at or near the time of data entry. This may be important with respect to certain health conditions that are affected, whether positively or negatively, through one or more factors external to the health product under evaluation. As such, the user may wish to enter these external factors as data, such that these factors are accounted for in self-assessment of progression or alleviation of symptoms.

For example, gastrointestinal health conditions may be negatively affected through the consumption of unusual foods and therefore ingestion of food outside the residence (i.e., “eating out”) may affect symptom condition. As another example, respiratory health conditions may be negatively affected by air quality or introduction to certain allergens (e.g., pet dander, pollen). As another example, the user experience may be selected from ingestion of a specific type of food, ingestion of food outside residence, travel, use of another health care product, stressful condition, failure to administer the health care product, menstruation, and combinations thereof.

The compliance aid can further comprise information related to use of the compliance aid, wherein the information comprises direction to a user to self-assess and self-determine an appropriate health care product to administer based on visual review of a plurality of entries of the symbolic data in the symbolic data entry fields.

Efficacy of certain health care products may commence only after a period of time, for example weeks or months. In one embodiment of the invention herein, information encouraging continued use of the product is provided to assist with, or prevent, premature termination of use of the health care product.

To illustrate, in a 28 day regimen of a health care product, the following information may be provided:
Week 1 tip:
What to expect: In the first few days of taking this health care product, some people may notice a temporary increase in bloating. This is normal and should go away soon. Your body is adjusting to healthy bowel function.
Week 2 tip:
What to expect: If you've been taking this product daily, you should be having fewer days when digestive upsets are adversely affecting your lifestyle. Keep on taking the product.
Week 3:
What to expect: You're well on your way to a noticeable improvement in your digestive health.
Week 4:
What to expect: Now that you're experiencing balanced digestive health, continue to take the product once a day, every day, to keep your system strong and healthy.

Alternatively, to illustrate, in a 14 day regimen of a health care product, the following information may be provided:
Week 1
What to expect: In the first few days of taking Align Advanced Daily Digestive Care, you should expect to see some differences in your digestive system. Some people notice a minor increase in gas or bloating. Others notice different signals that it is time to go, or changes to the form of their stool. These are all signs that your body is adjusting to healthy digestive function. Remember, the path to digestive health is a journey—stick with Align and you'll be on your way to freedom from digestive worries.
Week 2
What to expect: You should be experiencing a noticeable improvement in your digestive health—in fact, other people may be noticing that you are doing something new. Your body is well on your way to the full benefits of Align—continuing to take Align once a day, every day will help build your maximum defense against digestive upsets, and keep your system strong and healthy. Be sure to pay close attention to how you feel this week so you are ready to choose your next box of Align—you won't want to miss a day of healthy digestive balance!

### Selection and Product Indicia of the Present Invention

The present invention also comprises selection indicia and product indicia which are useful to enable a user to self-assess a health condition and select one or more appropriate health care products.

### Selection Indicia

The selection indicia relate to type, severity and frequency of symptoms associated with a health condition to be treated by a health care product. Selection indicia can be used by a potential user to self-assess a user's health condition, to educate a potential user with respect to a health condition, and aid a user in selecting an appropriate health care product for use with a health condition. Selection indicia can be associated with educational material, a compliance aid, sales displays and/or product packaging.

The form of the selection indicia may be dependent upon the form of the health care product and/or packaging or containment device containing the health care product. For example, wherein packaging containing a health care product is in the form of a cardboard or paperboard carton or box, the selection indicia may be in the form of a grid, chart, checklist or other means of self-assessment printed on the outside of the carton or box. A potential user can view the grid, chart or checklist to self-assess the user's health condition and select an appropriate health care product to use.

For example, selection indicia may include descriptions or representations of various symptoms, such as upset stomach, diarrhea, and constipation, and their severity and frequency and can include direction to a potential user to use a particular health care product based on the symptoms, severity, and frequency thereof, which may be visualized by a potential user. As the selection indicia is symbolic, interpretation and therefore self-assessment of health condition and self-determination of appropriate health care product to use is readily visualized.

“Symbolic”, as used herein, means indicia that visually represents symptoms without reliance on full text sentences or moderate to long phrases (e.g., phrases having more than about four words). In one embodiment, at least a portion of the selection indicia is selected from alphanumeric indicia (i.e., indicia containing both letters and numbers), alphabetic indicia (i.e., indicia containing letters), numeric indicia (i.e., data containing numbers), color indicia (i.e., indicia represented in a color or a combination of colors), variable symbolic indicia (e.g., check marks, “X” marks, slash marks, asterisks, punctuation marks (e.g., “?”, “!”, and the like), geometric symbols, pictures, and the like), and any combinations thereof. In yet another embodiment, at least a portion of the selection indicia is selected from numeric indicia, color indicia, variable symbolic indicia, and any combinations thereof. In yet another embodiment, the selection indicia is color indicia. In yet another embodiment, the selection indicia is geometric symbol indicia. In yet another embodiment, the selection indicia is variable symbolic indicia.

By way of non-limiting example selection indicia can include a chart with rows of text describing upset stomach, diarrhea and constipation; frequency of once per day, once per week, occasionally and the like; and severity, with color coded symbols in columns corresponding to one of two product choices depending on the type, frequency and severity of symptoms. Orange or red can be used to denote high frequency and severity of symptoms and green can be used to denote lower frequency and severity of symptoms. A user matches symptoms, frequency and severity to the color coded symbols to self-assess and determine an appropriate product to use.

### Product Indicia

Product indicia are used to identify a health care product and its characteristics. Product indicia can correspond to the selection indicia, and can also correspond to indicia on a compliance aid. Product indicia is used by a potential user to identify a health care product and aid a user in selecting an appropriate health care product for use with a health condition. Product indicia can be associated with educational material, a compliance aid, sales displays and/or product packaging.

The form of the product indicia may be dependent upon the form of the health care product and/or packaging or containment device containing the health care product. For example, wherein packaging containing a health care product is in the form of a cardboard or paperboard carton or box, the product indicia may be in the form of a color printed on the outside of the carton or box. A potential user can view the color which would correspond to selection indicia comprising color in a grid, chart or checklist to enable a user, after self-assessment of the user's health condition using the selection indicia, and select an appropriate health care product to use.

For example, product indicia may include various colors or shades of colors. A potential user can use the product indicia to identify a health care product selected by a potential user by way of the selection indicia. As the product indicia is symbolic, interpretation and therefore self-assessment of health condition and self-determination of appropriate health care product to use is readily visualized.

“Symbolic”, as used herein, means indicia that visually represents symptoms without reliance on full text sentences or moderate to long phrases (e.g., phrases having more than about four words). In one embodiment, at least a portion of the product indicia is selected from alphanumeric indicia (i.e., indicia containing both letters and numbers), alphabetic indicia (i.e., indicia containing letters), numeric indicia (i.e., data containing numbers), color indicia (i.e., indicia represented in a color or a combination of colors), variable symbolic indicia (e.g., check marks, “X” marks, slash marks, asterisks, punctuation marks (e.g., “?”, “!”, and the like), geometric symbols, pictures, and the like), and any combinations thereof. In yet another embodiment, at least a portion of the product indicia is selected from numeric indicia, color indicia, variable symbolic indicia, and any combinations thereof. In yet another embodiment, the product indicia is color indicia. In yet another embodiment, the product indicia is geometric symbol indicia. In yet another embodiment, the product indicia is variable symbolic indicia.

By way of non-limiting example, red or orange color can be printed or appear on a containment device containing a health care product for use with most frequent and/or severe symptoms, and green can be printed or appear on a containment device containing a health care product for use with less frequent and less severe symptoms.

### Kits of the Present Invention

In one embodiment, the invention is directed to kits comprising the compliance aid and a health care product. Both the compliance aid and the health care product have been described herein above.

In one embodiment, the compliance aid is integrated into a containment device containing the health care product. For example, the compliance aid may be reversibly adhered to a bottle, blister pack, or other containment device containing the health care product. As another example, the compliance aid and the health care product may be more loosely co-packaged as a pamphlet together with the product contained within a bottle, blister pack, or other containment device (for example, the compliance aid (as the pamphlet) may be contained within the bottle or other containment device or perhaps contained within the same shrinkwrap package). As yet another example, compliance aid and health care product may be contained as discrete or integrated components within a box or other like containment device. As yet another example, the compliance aid may be printed directly on a blister pack, bottle, or other containment device containing the health care product. Mode of co-packaging is not critical to the kits, but rather convenient association or ready availability of the two components may be desirable to the user.

To illustrate, the kit may comprise the compliance aid and a starter regimen of a particular health care product, provided to users through a health care professional (such as a pharmacist, nurse, physician, veterinarian, or managed care organization).

In another embodiment, the invention is directed to kits comprising selection indicia, product indicia, a health care product and a compliance aid. The selection indicia, product indicia, compliance aid and health care product have been described herein above.

In such an embodiment, selection indicia are integrated into a containment device containing a health care product. For example, the selection indicia may be reversibly adhered to a bottle, blister pack, or other containment device containing the health care product. As another example, the selection indicia may be printed directly on a box, carton, blister pack, bottle, or other containment device containing the health care product. In such an embodiment, product indicia may be incorporated integrally into a containment device containing the health care product. Mode of packaging is not critical to the kits, but rather convenient association of selection indicia, product indicia and product for self-assessment and selection of kit and product may be desirable to the user.

In another embodiment the invention is directed to a kit comprising selection indicia, product indicia, multiple health care products, and a compliance aid. The multiple health care products can be products complimentary to each other. Non-limiting examples of such multiple and complimentary health care products include a probiotic and fiber, and a probiotic and one or more vitamins.

### Methods of the Present Invention

In one embodiment, the invention is directed to methods of enhancing user compliance with a health care product useful for treatment of a health condition, comprising:
(a) providing a user a reference to the health care product;
(b) directing the user to use a compliance aid comprising a plurality of symbolic data entry fields, wherein the direction comprises:
   i. direction to, on a periodic basis, enter symbolic data into at least one of the symbolic data entry fields, wherein entry of the symbolic data is based on perceived symptoms of the health condition of the user at or near the time of data entry; and
   ii. direction to self-assess treatment of the health condition based on visual review of a plurality of entries of the symbolic data in the symbolic data entry fields.

In another embodiment, the method can also include direction to use and administer a particular health care product, products, or combination thereof based on a user's self-assessment of a health condition and/or treatment thereof.

These steps have been described above with respect to the compliance aid. In certain embodiments, the methods further comprise administration of the health care product to the user. As has been stated, the users herein include, but are not limited to, humans. The human user is often the user of the compliance aid and of the health care product referenced by the compliance aid. However, it will be recognized that a human may use the compliance aid, but for the purpose of tracking progression or alleviation of symptoms experienced by a non-human animal, such as a domestic animal (e.g., a dog, cat, rabbit, horse, or the like). In such instances, the user of the compliance aid is still the human, while the non-human animal experiences the health condition.

As used herein, the term “administration” or the like with respect to the user means that the user is administered, is directed to administer or, with reference specifically to “oral administration,” or “orally administering,” ingests or is directed to ingest, the health care product. For example, the administration may be oral administration, parenteral administration, topical administration, buccal administration, rectal administration, or the like, but will be dependent upon the nature of the health care product itself. In one embodiment the administration is oral administration.

Wherein the user is directed to administer the health care product, such direction may be that which instructs and/or informs the user that use of the composition may and/or will provide one or more general health and/or general physiological benefits associated with the health care product. For example, such direction may be oral direction (e.g., through oral instruction from, for example, a physician, health professional, sales professional or organization, and/or radio or television media (i.e., advertisement) or written direction (e.g., through written direction from, for example, a physician or other health professional (e.g., scripts), sales professional or organization (e.g., through, for example, marketing brochures, pamphlets, or other instructive paraphernalia), written media (e.g., internet, electronic mail, or other computer-related media), and/or containing devices associated with the composition (e.g., a label present on a package containing the composition). See e.g., the kits described herein.

In an alternative embodiment, the invention is directed to methods of selecting and administering a health care product comprising the steps of:
a. providing selection indicia for selection of a health care product;
b. providing corresponding product indicia associated with a containment device containing a health care product, wherein a potential user matches selection indicia and product indicia to select an appropriate health care product; and
c. administering the health care product selected.

The method can also include a compliance aid to track a user's progress and/or provide direction to a user regarding an appropriate health care product to administer and use.

### NON-LIMITING EXAMPLES

### Example 1

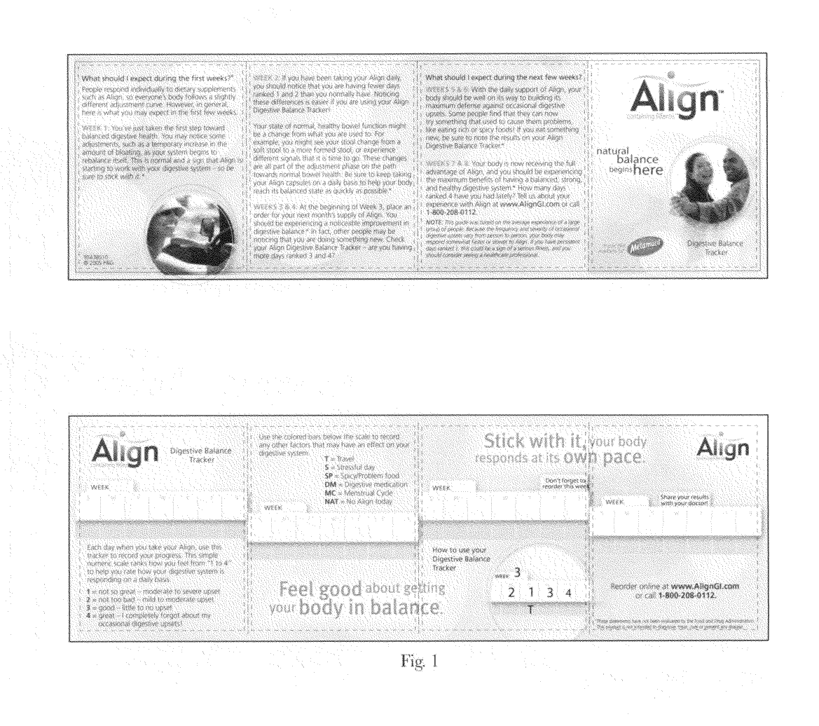

A non-limiting example of the present invention is illustrated at FIG. 1.

Reference to Health Care Product. The compliance aid contains a reference to a health care product (in this illustration, ALIGN® (containing a bacterial microorganism (e.g., BIFANTIS®)).

Plurality of Symbolic Data Entry Fields. The compliance aid comprises 28 symbolic data entry fields corresponding with a 28 day period of time (and visually separated into 4 one-week periods of time).

Information Related to Use of the Compliance Aid. The compliance aid comprises information related to use of the compliance aid.
Direction to a user to, on a periodic basis, enter symbolic data into at least one of the symbolic data entry fields, wherein entry of the symbolic data is based on perceived symptoms of the health condition at or near the time of data entry. “Each day when you take your Align, use this tracker to record your progress. This simple numeric scale ranks how you feel from “1 to 4” to help you rate how your digestive system is responding on a daily basis.
1=not so great—moderate to severe upset
2=not so bad—mild to moderate upset
3=good—little to no upset
4=great—I completely forgot about my occasional digestive upsets!”
Direction to a user to self-assess treatment of the health condition based on visual review of a plurality of entries of the symbolic data in the symbolic data entry fields. “This simple numeric scale ranks how you feel from “1 to 4” to help you rate how your digestive system is responding on a daily basis.”

### Example 2

A non-limiting example of the present invention is illustrated at FIG. 2.

Reference to Health Care Product. The compliance aid contains a reference to a health care product (in this illustration, ALIGN® (containing a bacterial microorganism (e.g., BIFANTIS®)).

Plurality of Symbolic Data Entry Fields. The compliance aid comprises 28 symbolic data entry fields corresponding with a 28 day period of time (and visually separated into 4 one-week periods of time), wherein each symbolic data entry field contains 5 symbolic data entry sub-fields. Among each of the 5 symbolic data entry sub-fields, 4 of the symbolic data entry sub-fields (in this example, color bars) are designated such that the user chooses one of these sub-fields for data entry based upon perceived symptoms of the health condition. The remaining symbolic data entry sub-field (in this example, “the white bar”) is designated, at the discretion of the user, for entry of symbolic data (in this example, alphabetic symbolic data (i.e., “SP”, “S”, “T”, “SM”, “MC”, “MA”)) based on a user experience at or near the time of data entry.

Information Related to Use of the Compliance Aid. The compliance aid comprises information related to use of the compliance aid.
Direction to a user to, on a periodic basis, enter symbolic data into at least one of the symbolic data entry fields, wherein entry of the symbolic data is based on perceived symptoms of the health condition at or near the time of data entry. “EVERY DAY: Use the “How Do I Feel” criteria to record your progress. Each day when you take your Align capsule, put an “X” in the color bar that matches how you feel. Because foods, stress, travel, medications, etc., can trigger or intensify digestive upsets, use the white bar to record any “triggers” that may apply.”

In this example, the symbolic data entry fields are color coded as color bars, wherein the color bars are arranged in 28 separate columns corresponding to the 28 symbolic data entry fields. Each color bar within a symbolic data entry field is a symbolic data entry sub-field and are described as follows, arranged in each column from highest to lowest:
Blue=“Great!—I completely forgot about my digestive upsets!”
Green=“Good—few if any upsets”
Yellow=“Occasionally Bothered—mild to moderate upsets”
Orange=“Often Bothered—moderate to severe upsets”
   (Note that the lowest color bar in the column, in this instance the “white” bar, is a symbolic data entry sub-field for entry based on a user experience at or near the time of data entry.)

Since within a given columnar symbolic data entry field the symbolic data entry sub-fields are arranged in descending order of “Blue”, “Green”, “Yellow”, and “Orange”, alleviation of symptoms over time will be visually self-assessed by observing a general trend of rising “X” marks (symbolic data) over time, as is demonstrated in FIG. 2. Similarly, progression of symptoms over time will be visually self-assessed by observing a general trend of descending “X” marks over time, while generally no significant trends in symptoms will be visually self-assessed by observing no general trend of “X” marks over time.
Direction to a user to self-assess treatment of the health condition based on visual review of a plurality of entries of the symbolic data in the symbolic data entry fields.
   “Daily Digestive Tracker . . . . Monitor your progress over the next 4 weeks” “If you have persistent days rated “orange,” this could be a sign of serious illness, and you should consider seeing a healthcare professional.”
   “EVERY WEEK: With every week, you should see more “X's” in the green and blue color bars—a sure sign of your progress! But remember, everyone responds to Align at a different pace, so be patient and keep taking Align for best results.”

### Example 3

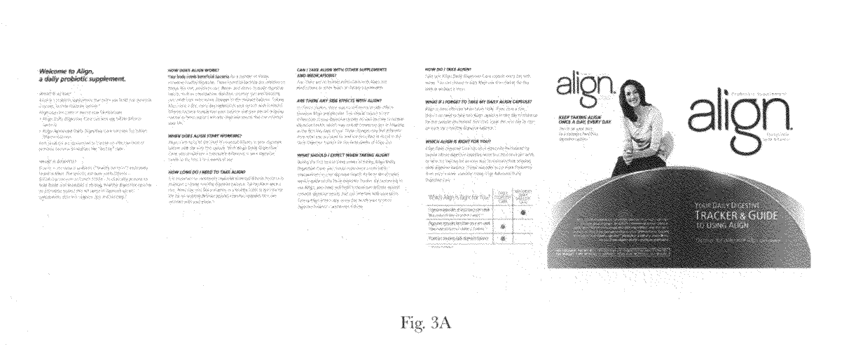

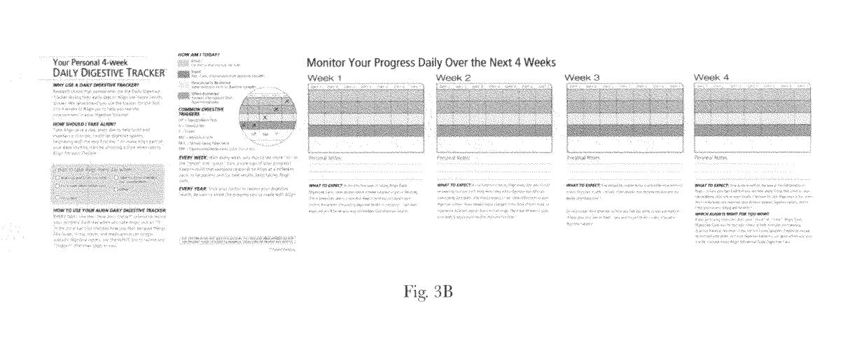

A non-limiting example of the present invention is illustrated at FIGS. 3a and 3b which represent the front and back, respectively, of a compliance aid.

Reference to Health Care Product. The compliance aid contains a reference to a health care product (in this illustration, ALIGN® (containing a bacterial microorganism (e.g., BIFANTIS®)).

### Selection Indicia

WHICH ALIGN IS RIGHT FOR YOU?

“Align Daily Digestive Care has been especially formulated for people whose digestive episodes occur less than once per week, or who are looking for an easy way to maintain their ongoing daily digestive balance. If your episodes occur more frequently than once a week, consider trying Align Advanced Daily Digestive Care.”

A chart listing criteria and product choice follows, with Align Daily Digestive Care accompanied by Green color indicia and Align Advanced Daily Digestive Care accompanied by Orange color indicia.

### Product Indicia

The compliance aid contains color indication that corresponds to the selection indicia. In this example, the background color for the Daily Digestive Tracker and Guide to Using Align is Green, to accompany Align Daily Digestive Care product.

Plurality of Symbolic Data Entry Fields. The compliance aid comprises 28 symbolic data entry fields corresponding with a 28 day period of time (and visually separated into 4 one-week periods of time).

Information Related to Use of the Compliance Aid. The compliance aid comprises information related to use of the compliance aid.
Direction to a user to, on a periodic basis, enter symbolic data into at least one of the symbolic data entry fields, wherein entry of the symbolic data is based on perceived symptoms of the health condition at or near the time of data entry. “Use the “How am I today?” criteria to record your progress. Each day when you take Align, put an “X” in the color bar that matches how you feel. Because things like food, stress, medications, and travel can trigger episodic digestive upsets, use the WHITE bar to record any “triggers” that may apply to you.”
How am I today?
Orange=Often Bothered—frequent interruptions from digestive episodes
Yellow=Occasionally Bothered—some interruptions from digestive episodes
Green=Good—few, if any, interruptions from digestive episodes
Blue=Great—I'm free to plan my day my way!”
Direction to a user to self-assess treatment of the health condition based on visual review of a plurality of entries of the symbolic data in the symbolic data entry fields.

WHICH ALIGN IS RIGHT FOR YOU NOW?

“If you are having consistent days rated “Good” or “Great”, Align Daily Digestive Care may be the right choice to help maintain your ongoing digestive balance. However, if you are still having episodes frequently enough to interrupt your plans, or if you digestive balance is not quite where you wan it to be, consider trying Align Advanced Daily Digestive Care.”

### Example 4

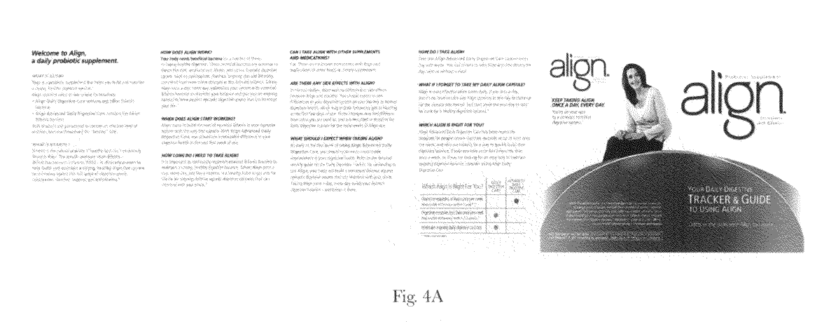

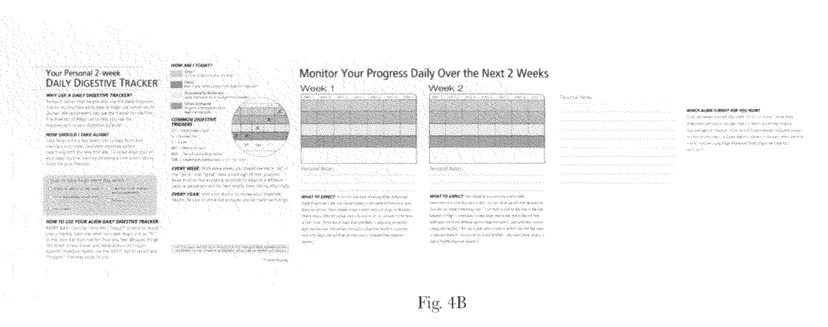

A non-limiting example of the present invention is illustrated at FIGS. 4a and 4b which represent the front and back, respectively, of a compliance aid.

Reference to Health Care Product. The compliance aid contains a reference to a health care product (in this illustration, ALIGN® (containing a bacterial microorganism (e.g., BIFANTIS®)).

### Selection Indicia

WHICH ALIGN IS RIGHT FOR YOU?

“Align Advanced Daily Digestive Care has been especially formulated for people whose digestive episodes occur at least once per week, and who are looking for a way to quickly build their digestive balance. If your episodes occur less frequently than once a week, consider trying Align Daily Digestive Care.”

A chart listing criteria and product choice follows, with Align Daily Digestive Care accompanied by Green color indicia and Align Advanced Daily Digestive Care accompanied by Orange color indicia.

### Product Indicia

The compliance aid contains color indication that corresponds to the selection indicia. In this example, the background color for the Daily Digestive Tracker and Guide to Using Align is Orange, to accompany Align Advanced Daily Digestive Care product.

Plurality of Symbolic Data Entry Fields. The compliance aid comprises 14 symbolic data entry fields corresponding with a 14 day period of time (and visually separated into 2 one-week periods of time).

Information Related to Use of the Compliance Aid. The compliance aid comprises information related to use of the compliance aid.
Direction to a user to, on a periodic basis, enter symbolic data into at least one of the symbolic data entry fields, wherein entry of the symbolic data is based on perceived symptoms of the health condition at or near the time of data entry. “Use the “How am I today?” criteria to record your progress. Each day when you take Align, put an “X” in the color bar that matches how you feel. Because things like food, stress, medications, and travel can trigger episodic digestive upsets, use the WHITE bar to record any “triggers” that may apply to you.”
How am I today?
Orange=Often Bothered—frequent interruptions from digestive episodes
Yellow=Occasionally Bothered—some interruptions from digestive episodes
Green=Good—few, if any, interruptions from digestive episodes
Blue=Great—I'm free to plan my day my way!”
Direction to a user to self-assess treatment of the health condition based on visual review of a plurality of entries of the symbolic data in the symbolic data entry fields.

WHICH ALIGN IS RIGHT FOR YOU NOW?

“If you are having consistent days rated “Good” or “Great”, Align Daily Digestive Care may be the right choice to help maintain your ongoing digestive balance. However, if you are still having episodes frequently enough to interrupt your plans, or if you digestive balance is not quite where you wan it to be, consider trying Align Advanced Daily Digestive Care for 2 more weeks.”

### Example 5

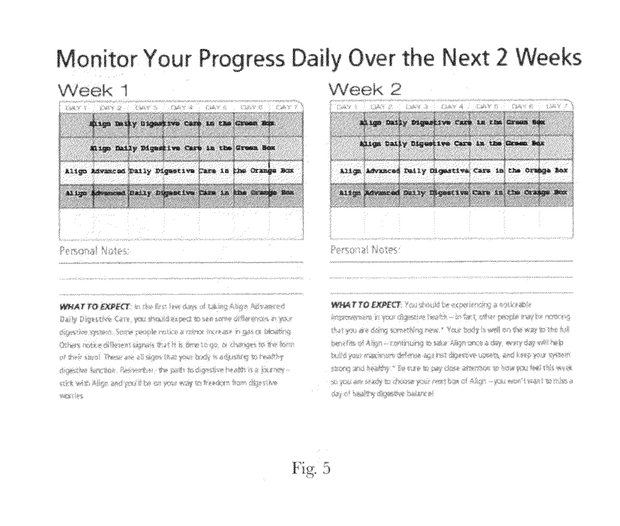

A non-limiting example of the present invention is illustrated at FIG. 5.

FIG. 5 illustrates a portion of a compliance aid. The portion illustrated contains selection indicia.

Plurality of Symbolic Data Entry Fields. The compliance aid comprises 14 symbolic data entry fields corresponding with a 14 day period of time (and visually separated into 2 one-week periods of time).

Information Related to Use of the Compliance Aid. The compliance aid comprises information related to use of the compliance aid.
Direction to a user to self-assess treatment of the health condition based on visual review of a plurality of entries of the symbolic data in the symbolic data entry fields. Within each color bar is selection indicia in the form of text. In the Blue (Great) and Green (Good) color bars is the direction: “Align Daily Digestive Care in the Green Box”. In the Yellow (Occasionally Bothered) and Orange (Often Bothered) color bars is the direction: “Align Advanced Daily Digestive Care in the Orange Box”.

### Example 6

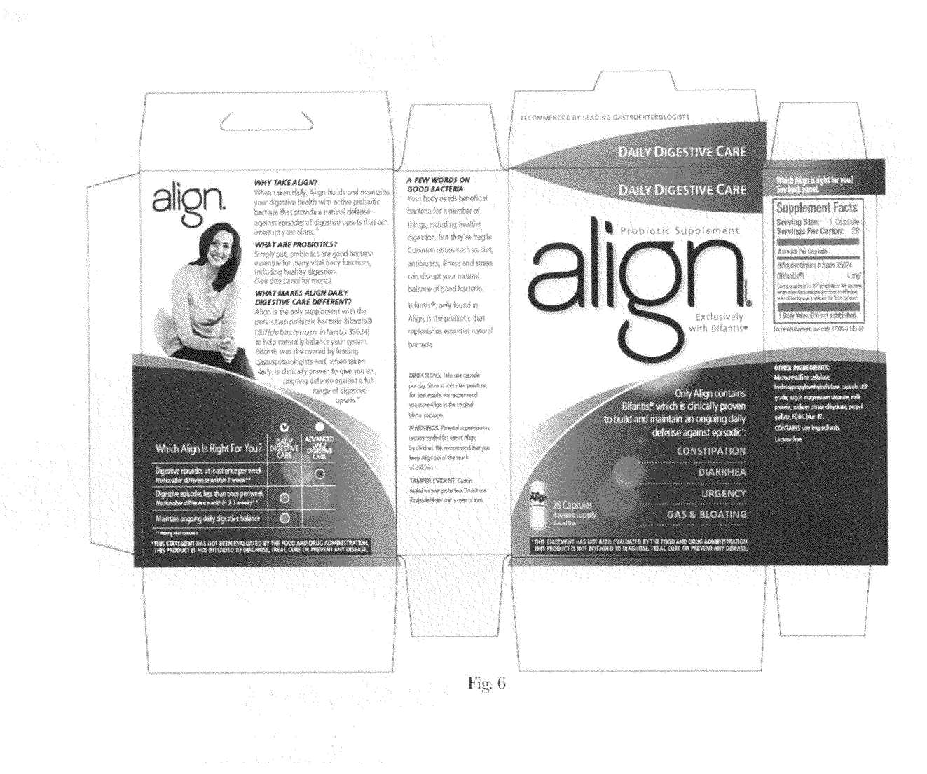

A non-limiting example of the present invention is illustrated at FIG. 6, which shows a containment device for a health care product, in un-folded form prior to cutting, folding, and sealing such that all portions of the containment device are visible in the Figure.

Reference to Health Care Product. The containment device contains a reference to a health care product (in this illustration, ALIGNS® (containing a bacterial microorganism (e.g., BIFANTIS®)).

### Selection Indicia

The containment device contains selection indicia to aid a potential user in selecting an appropriate health care product.

WHICH ALIGN IS RIGHT FOR YOU?

A chart listing criteria and product choice follows, with Align Daily Digestive Care accompanied by Green color indicia and Align Advanced Daily Digestive Care accompanied by Orange color indicia is presented on the containment device.

### Product Indicia

The containment device contains color indicia that corresponds to the selection indicia. In this example, the background color for the containment device, headings of text, and other indicia thereon are colored Green to accompany Align Daily Digestive Care product.

Thus, a potential user can self-assess his or her health condition based on review of the selection indicia and choose an appropriate health care product based on the selection indicia and corresponding product indicia.

### Example 7

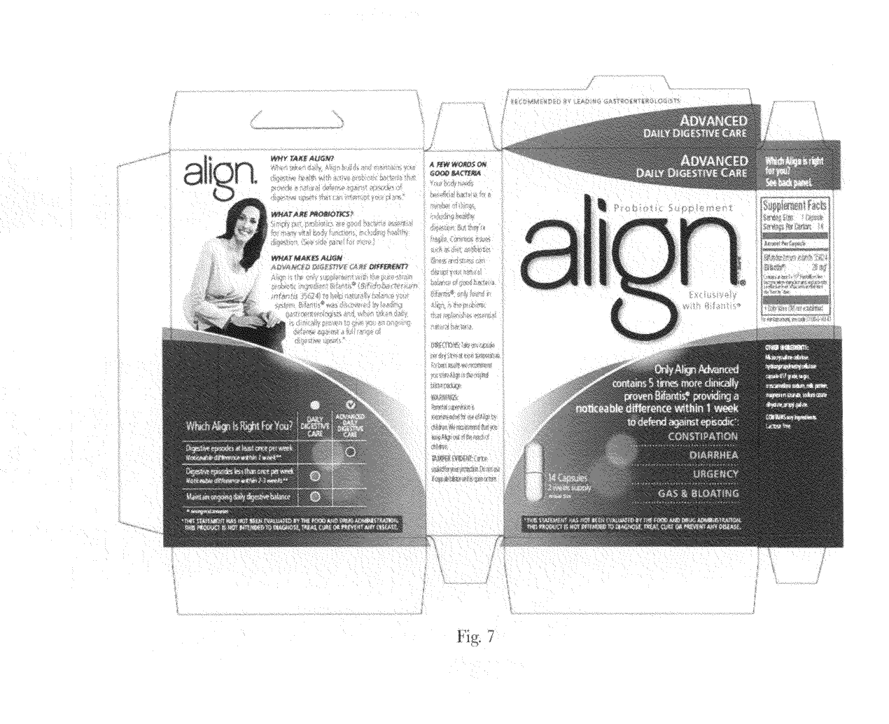

A non-limiting example of the present invention is illustrated at FIG. 7, which shows a containment device for a health care product, in un-folded form prior to cutting, folding, and sealing such that all portions of the containment device are visible in the Figure.

Reference to Health Care Product. The containment device contains a reference to a health care product (in this illustration, ALIGN® (containing a bacterial microorganism (e.g., BIFANTIS®)).

### Selection Indicia

The container contains selection indicia to aid a potential user in selecting the appropriate health care product.

WHICH ALIGN IS RIGHT FOR YOU?

A chart listing criteria and product choice follows, with Align Daily Digestive Care accompanied by Green color indicia and Align Advanced Daily Digestive Care accompanied by Orange color indicia is presented on the containment device.

### Product Indicia

The containment device contains color indication that corresponds to the selection indicia. In this example, the background color for the containment device, headings of text, and other indicia thereon are colored Orange to accompany Align Advanced Daily Digestive Care product.

Thus, a potential user can self-assess his or her health condition based on review of the selection indicia and choose an appropriate health care product based on the selection indicia and corresponding product indicia.

### Example 8

An adult female has been suffering from episodes of digestive upsets which occur once a week or more often and she would like to build and maintain a healthier digestive system. The woman investigates options for improving her overall digestive balance and finds kits containing a health care product that contains a probiotic. There are two choices of product, containing two levels of bacteria (for example ‘loading’ and ‘maintenance’ levels of bacteria). The product containment devices of the kits each contain selection indicia to enable the potential user to self-assess her health condition, and corresponding product indicia to enable the user to select an appropriate kit and health care product.

The containment device for the product for use with the most frequent and severe symptoms has orange product indicia as a part thereof. The containment device for the product for use with the less frequent and less severe symptoms has green product indicia as a part thereof. Each containment device also contains selection indicia comprising a chart or grid having rows of text describing frequency and effect of digestive upsets, including “digestive episodes at least once per week”, “digestive episodes less than once per week”, and “maintain ongoing daily digestive balance”. The grid also contains columns identifying each of two products, “Daily Digestive Care” and “Advanced Daily Digestive Care”. Green circles appear in the column “Daily Digestive Care”, next to the rows “digestive episodes less than once per week”, and “maintain ongoing daily digestive balance”. An orange circle appears in the column “Advanced Daily Digestive Care” and next to the row “digestive episodes at least once per week.”

She views the selection indicia and matches her symptom frequency and how quickly she would like to notice a difference in her digestive health to self-assess and identify the appropriate product for her health condition. In her case, her symptom frequency falls into the orange area of the selection indicia. She selects the “Advanced Daily Digestive Care” product based on self-assessment using the selection indicia. She is then easily able to identify the “Advanced Daily Digestive Care” product by the product indicia on the containment device containing the product. The corresponding product containment device for the product “Advanced Daily Digestive Care” product containing “loading” levels of bacteria has orange product indicia, such that she chooses the appropriate product. The appropriate product is easy to determine and select because the product containment device has product indicia thereon matching the selection indicia.

She administers the product for two weeks and uses the compliance aid included in the kit to track her progress. At the beginning of the two weeks, most of her entries fell into the orange area of the compliance aid, but gradually moved into the blue and the green areas as the two weeks progressed. After reviewing her progress, she decides that her next purchase will be the “Daily Digestive Care” product containing the ‘maintenance’ level of bacteria because she now has few, if any, interruptions from digestive episodes. The green and blue color indicia on the compliance aid help track her progress and direct her to select the appropriate next product. Green product indicia on the containment device of the “Daily Digestive Care” product enable her to easily find and choose the appropriate product.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mg” is intended to mean “about 40 mg”.

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of selecting and administering a health care product comprising the steps of:
a. providing selection indicia for selection of a health care product;
b. providing corresponding product indicia associated with a containment device containing said health care product, wherein a potential user matches said selection indicia and said product indicia to select an appropriate said health care product; and
c. administering said health care product selected.

2. The method of claim 1 wherein said selection indicia is associated with said containment device containing said health care product.

3. The method of claim 1 further comprising providing a compliance aid to track, assess and improve a user's use of and compliance with said method, and aid a user in selecting said appropriate health care product for continued use.

4. The method of claim 3 wherein said compliance aid comprises indicia corresponding to said selection and said product indicia.

5. The method of claim 1 wherein at least a portion of the selection indicia is selected from the group consisting of alphanumeric indicia, alphabetic indicia, numeric indicia, color indicia, variable symbolic indicia, and combinations thereof.

6. The method of claim 1 wherein at least a portion of the product indicia is selected from the group consisting of alphanumeric indicia, alphabetic indicia, numeric indicia, color indicia, variable symbolic indicia, and combinations thereof.

7. The method of claim 1 further wherein said selection indicia and said product indicia enable a user to select multiple appropriate health care products.

8. The method of claim 1 wherein said health care product is selected from the group consisting of gastrointestinal health care products, respiratory health care products, and wellness health care products.

9. The method of claim 1 wherein said health care product comprises a probiotic.

10. The method of claim 9 wherein said probiotic comprises lactic acid bacteria selected from the group consisting of Bifidobacterium, Lactobacillus, Streptococcus and combinations thereof.

11. The method of claim 10 wherein said lactic acid bacteria comprises an isolated strain of Bifidobacterium infantis.

12. The method of claim 9 further comprising administering said probiotic to provide from about 1×10³ to about 1×10¹⁴ cfu of probiotic per day.

13. The method of claim 12 further comprising administering said probiotic to provide from about 1×10⁵ to about 1×10¹⁴ cfu of probiotic per day.

14. The method of claim 13 further comprising administering said probiotic to provide from about 1×10⁹ to about 1×10¹² cfu of probiotic per day.

15. The method of claim 9 further comprising administering said probiotic to provide a from about 1×10³ to about 1×10¹² cfu of maintenance probiotic per day.

16. The method of claim 15 further comprising administering said probiotic to provide from about 1×10⁷ to about 1×10¹⁰ cfu of maintenance probiotic per day.

17. The method of claim 1 wherein said health care product is administered in a dosage form selected from the group consisting of: capsule, chewable tablet, swallowable tablet, buccal tablet, troche, powder, lozenge, soft chew, solution, suspension, spray, tincture, decoction, infusion, syrup, exlixir, wafer, food product, and combinations thereof.

18. A method of selecting and administering a health care product comprising the steps of:
a. providing selection indicia for selection of an appropriate health care product;
b. providing corresponding product indicia associated with a containment device containing said health care product, wherein a potential user matches said selection indicia and said product indicia to select said appropriate health care product;
c. administering said health care product selected;
d. providing a compliance aid to track, assess and improve a user's use of and compliance with said method, and aid a user in selecting an appropriate said health care product for continued use.

19. The method of claim 18 wherein said selection indicia is associated with said containment device containing said health care product.

20. The method of claim 18 wherein said compliance aid comprises indicia corresponding to said selection indicia and said product indicia.

21. The method of claim 18 wherein said health care product is selected from the group consisting of gastrointestinal health care products, respiratory health care products, and wellness health care products.

22. The method of claim 18 wherein said health care product comprises a probiotic.

23. The method of claim 22 wherein said probiotic comprises lactic acid bacteria selected from the group consisting of Bifidobacterium, Lactobacillus, Streptococcus and combinations thereof.

24. The method of claim 23 wherein said lactic acid bacteria comprises an isolated strain of Bifidobacterium infantis.

25. The method of claim 22 further comprising administering said probiotic to provide from about 1×10³ to about 1×10¹⁴ cfu of probiotic per day.

26. The method of claim 25 further comprising administering said probiotic to provide from about 1×10⁵ to about 1×10¹⁴ cfu of probiotic per day.

27. The method of claim 26 further comprising administering said probiotic to provide from about 1×10⁹ to about 1×10¹² cfu of probiotic per day.

28. The method of claim 22 further comprising administering said probiotic to provide from about 1×10³ to about 1×10¹² cfu of probiotic per day.

29. The method of claim 28 further comprising administering said probiotic to provide from about 1×10⁷ to about 1×10¹⁰ cfu of probiotic per day.

30. A kit for use in selecting and administering a health care product comprising:
a. selection indicia for selection of an appropriate health care product;
b. corresponding product indicia associated with said kit, wherein a potential user matches said selection indicia and said product indicia to select said appropriate kit;
c. a compliance aid to track, assess and improve a user's use of and compliance with using said health care product; and
d. a health care product.

31. The kit of claim 30 further comprising instructions for use of said kit.

32. The kit of claim 30 wherein said health care product is selected from the group consisting of gastrointestinal health care products, respiratory health care products, and wellness health care products.

33. The kit of claim 30 wherein said health care product comprises a probiotic.

34. The kit of claim 33 wherein said probiotic comprises lactic acid bacteria selected from the group consisting of Bifidobacterium, Lactobacillus, Streptococcus and combinations thereof.

35. The kit of claim 34 wherein said lactic acid bacteria comprises an isolated strain of Bifidobacterium infantis.

36. The kit of claim 30 further comprising an additional health care product.

37. A method of selecting and administering a probiotic comprising the steps of:
a. providing selection indicia for selection of a probioitc;
b. providing corresponding product indicia associated with a containment device containing said probiotic, wherein a potential user matches said selection indicia and said product indicia to select an appropriate said probiotic; and
c. administering said probiotic selected.

38. A kit for use in selecting and administering a probiotic comprising:
a. selection indicia for selection of an appropriate probiotic;
b. corresponding product indicia associated with said kit, wherein a potential user matches said selection indicia and said product indicia to select an appropriate said kit;
c. a compliance aid to track, assess and improve a user's use of and compliance with using said probiotic; and
d. a probiotic.

39. The kit of claim 38 further comprising a health care product.
